(19) United States
(12) Patent Application Publication
(10) Pub. No.: US 2010/0318011 A1
(43) Pub. Date: Dec. 16, 2010

(54) LUMBAR BRACE FOR PREVENTING LUMBAR KYPHOSIS

(76) Inventors: Kashichi HIROTA, Hachioji-shi (JP); Junji FUKUDA, Chiba-shi (JP)

Correspondence Address: JACOBSON HOLMAN PLLC, 400 SEVENTH STREET N.W., SUITE 600, WASHINGTON, DC, 20004, US

(21) Appl. No.: 12/814,891

(22) Filed: Jun. 14, 2010

(30) Foreign Application Priority Data
Jun. 16, 2009 (JP) 2009-143167
Oct. 14, 2009 (JP) 2009-236853

(51) Int. Cl. A61F 5/01

(52) U.S. Cl. 602/19

(57) ABSTRACT

Provided is a lumbar kyphosis-preventing lumbar brace which is capable of being placed on a back surface of a lumbar portion of a wearer to provide resistance to lumbar kyphosis, while facilitating a wearing operation thereof without the occurrence a displacement when it is being worn or during use. The lumbar kyphosis-preventing lumbar brace comprises: a generally rectangular-shaped resilient plate which has a width dimension capable of covering a lateral region of a back surface of a lumbar portion of a wearer, and a height dimension capable of covering between a hip region and a lower back region of the back surface of the lumbar portion; and a belt engagement member provided on one side of the resilient plate in an engageable manner with a belt of a garment worn by the wearer. The lumbar brace is adapted to be worn in such a manner that the resilient plate is placed on the side of a wearer's body with respect to the garment, while allowing the belt engagement member to be located outside the garment and engaged with the belt of the garment. For example, the resilient plate is made of a resin material, an animal hide such as a cattle hide, or a rubber material.

### BACKGROUND OF THE INVENTION

1. Field of the Invention

The present invention relates to a lumbar brace for preventing lumbar kyphosis (an outward rounding or curving of a lumbar region of a spine). More specifically, the present invention relates to a lumbar kyphosis-preventing lumbar brace which is adapted to be placed on a back surface of a lumbar portion of a wearer to provide resistance to lumbar kyphosis.

2. Description of the Background Art

Recent years, many people suffer from lower back pain. One possible cause would be that aging gives rise to a tendency for the lumbar portion to naturally round or curve. Heretofore, as a brace for protecting a lumbar portion of a person, there have been known corsets as described in the following Patent Documents 1 to 4. All of the corsets are designed to be used by a person having low back pain but not to be worn for the purpose of preventing lumbar kyphosis. Moreover, this conventional type of corset is poor in usability and uncomfortable to wear.

[Patent Document 1] JP 3-65178B

[Patent Document 2] JP 2585656B

[Patent Document 3] JP 2870691B

[Patent Document 4] JP 3337358B

### SUMMARY OF THE INVENTION

It is an object of the present invention to provide a lumbar kyphosis-preventing lumbar brace capable of being placed on a back surface of a lumbar portion of a wearer to provide resistance to lumbar kyphosis, while facilitating a wearing operation thereof without the occurrence a displacement when it is being worn or during use.

In order to achieve the above object, the present invention provides a lumbar kyphosis-preventing lumbar brace which comprises: a generally rectangular-shaped resilient plate made of a resin material, an animal hide such as a cattle hide, a molded rubber material, or a laminated material prepared by laminating a plurality of sheets made of one or more of the materials; and a belt engagement member. For example, the resilient plate may be a plate made of only one of the above materials. Alternatively, the resilient plate may be a laminated plate prepared by laminating an animal hide such as a cattle hide to one surface or both surfaces of a core layer made of a plate-shaped resin material, rubber material or board. The resilient plate has a width dimension capable of covering a lateral region of a back surface of a lumbar portion of a wearer, and a height dimension capable of covering between a hip region and a lower back region of the back surface of the lumbar portion. The belt engagement member is provided on one side of the resilient plate, and may have a belt engagement portion engageable with a belt of a garment worn by the wearer. The lumbar brace is adapted to be worn in such a manner that the resilient plate is placed on the side of a wearer's body with respect to the garment, while allowing the belt engagement member to be located outside the garment and engaged with the belt of the garment. Thus, when the wearer buckles the belt, the resilient plate is brought into contact with the back surface of the lumbar portion of the wearer in the range covering between the hip region and the lower back region to provide a resistance force against a forward bending (flexion) of the lumbar portion. Consequently, the wearer naturally acquires a habit of stretching the lumber portion, which makes it possible to prevent lumbar kyphosis. In other words, in the present invention, the term “resilient” or “resiliency” is used to express a physical property capable of providing a resistance force against a forward bending (flexion) of a lumbar portion of a wearer.

The belt engagement member may be formed integrally with the resilient plate. Alternatively, the belt engagement member may be formed separately from the resilient plate, and detachably attached to the resilient plate.

In cases where the belt engagement member is formed separately from the resilient plate, the resilient plate may have an engagement member-receiving hole which is formed in a protruding portion on one surface thereof and opened in an upward-downward direction, wherein the belt engagement member may has a leg portion insertable into the engagement member-receiving hole, whereby the belt engagement member can be connected to the resilient plate by inserting the leg portion into the engagement member-receiving hole. Alternatively, the resilient plate may have an engagement member-receiving hole formed to penetrate therethrough in a thicknesswise direction thereof, wherein the belt engagement member may have a leg portion formed with a pin insertable into the engagement member-receiving hole, whereby the belt engagement member can be connected to the resilient plate by inserting the leg portion into the engagement member-receiving hole of the resilient plate. In either case, it is preferable that the belt engagement member is disposed in a widthwise central region of the resilient plate in the widthwise direction. Alternatively, a plurality of the belt engagement members may be arranged at respective widthwise positions of the resilient plate. Further, the belt engagement member may be designed such that it is formed in a loop or ring shape, and one end thereof is attached to the resilient member by a rivet. In this case, the loop or ring-shaped belt engagement member may be attached to the resilient plate in a 360-degree rotatable relation. The belt engagement member may be designed such that it is formed as a strip-like member made of a resin material such as polypropylene, and a thin-walled portion is formed in an intermediate region of the strip-like member to extend in a widthwise direction thereof and serve as a bendable portion, wherein one end of the strip-like member is connected to the resilient plate, and then bent inwardly along the thin-walled portion to form a loop portion for allowing the belt to penetrate therethrough.

Preferably, the resilient plate has a plurality of vent holes formed to penetrate therethrough in a thicknesswise direction thereof. Preferably, the resilient plate has a cloth attached onto at least a surface thereof facing the wearer's body, to improve feel during use. The resilient plate may be put in a bag-like cloth cover with a fastener. In cases where the resilient plate is formed of an animal hide such as a cattle hide, or a rubber material, it is preferably to put the resilient plate in the cloth cover, particularly, in view of external appearance.

The resilient plate may have one or more magnetic therapeutic devices each equipped with a permanent magnet piece and provided on a surface thereof facing the wearer's body.

As above, the lumbar brace is adapted to be worn in such a manner that the resilient plate is placed on the side of the wearer's body with respect to the garment, while allowing the belt engagement member to be located outside the garment and engaged with the belt of the garment, which makes it possible to facilitate a wearing operation thereof In addition, through the wearing operation, the belt engagement member is engaged with the belt of the garment to allow the resilient plate to be held by the belt, which makes it possible to prevent the occurrence of a displacement of the resilient plate during use. Further, in a state after the belt is buckled, the resilient plate is brought into contact with the back surface of the lumbar portion of the wearer in the range covering between the hip region and the lower back region to provide a resistance force against a forward bending (flexion) of the lumbar portion, which makes it possible to prevent the forward bending (flexion) of the lumbar portion and obtain an effective preventative effect against lower back pain. This lower back pain-preventing effect can be enhanced by the structure where the one or more magnetic therapeutic devices each having a permanent magnet piece is provided on the resilient plate.

### BRIEF DESCRIPTION OF DRAWINGS

FIG. 1 is a side view showing a state after a lumbar kyphosis-preventing lumbar brace according to one embodiment of the present invention is attached to trousers.

FIGS. 2(a) to 2(c) illustrate a structure of a lumbar kyphosis-preventing lumbar brace according to a first embodiment of the present invention, wherein FIG. 2(a) is a back view, when viewed from the side of a back surface of a resin plate, and FIG. 2(b) and FIG. 2(c) are a sectional view taken along the line B-B in FIG. 2(a) and a sectional view taken along the line C-C in FIG. 2(a), respectively.

FIGS. 3(a) to 3(c) illustrate a belt engagement member for use in the brace according to the first embodiment illustrated in FIGS. 2(a) to 2(c), wherein FIG. 3(a), FIG. 3(b) and FIG. 3(c) are a front view, a side view and a back view, respectively.

FIGS. 4(a) to 4(c) illustrate a structure of a lumbar kyphosis-preventing lumbar brace according to a second embodiment of the present invention, wherein FIG. 4(a) is a back view, when viewed from the side of a back surface of a resin plate, and FIG. 4(b) and FIG. 4(c) are a sectional view taken along the line B-B in FIG. 4(a) and a sectional view taken along the line C-C in FIG. 4(a), respectively.

FIG. 5 is a development view of a cloth for use in the second embodiment illustrated in FIGS. 4(a) to 4(c).

FIGS. 6(a) to 6(c) illustrate a structure of a lumbar kyphosis-preventing lumbar brace according to a third embodiment of the present invention, wherein FIG. 6(a) is a back view, when viewed from the side of a back surface of a resin plate, and FIG. 6(b) and FIG. 6(c) are a sectional view taken along the line B-B in FIG. 6(a) and a sectional view taken along the line C-C in FIG. 6(a), respectively.

FIGS. 7(a) and 7(b) illustrate a belt engagement member for use in the brace according to the third embodiment illustrated in FIGS. 6(a) to 6(c), wherein FIG. 7(a) is a front view, and FIG. 7(b) is a side view.

FIGS. 8(a) to 8(d) illustrate a structure of a lumbar kyphosis-preventing lumbar brace according to a fourth embodiment of the present invention, wherein FIG. 8(a) and FIG. 8(b) are a back view, when viewed from the side of a back surface of a resin plate, and a front view, respectively, and FIGS. 8(c) and 8(d) are a sectional view taken along the line C-C in FIG. 8(b) and a top plan view, respectively.

FIG. 9 is a sectional view showing another example of the belt engagement member.

FIG. 10 is a sectional view showing yet another example of the belt engagement member.

FIG. 11 is a front view showing a lumbar kyphosis-preventing lumbar brace according to a further embodiment of the present invention, wherein a resilient plate is put in a bag-like cloth cover with a fastener.

FIG. 12 is a sectional view of the embodiment illustrated in FIG. 11.

### DESCRIPTION OF THE PREFERRED EMBODIMENTS

With reference to the drawings, the present invention will be described based on various embodiments thereof

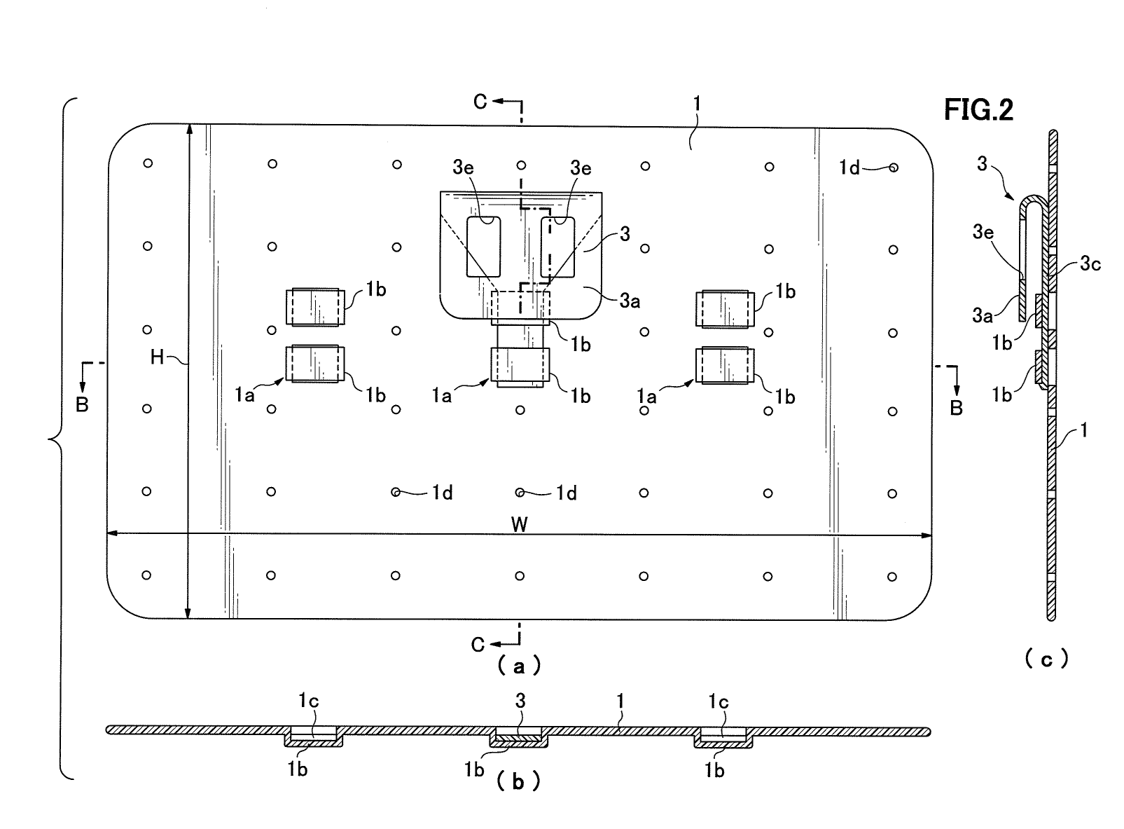

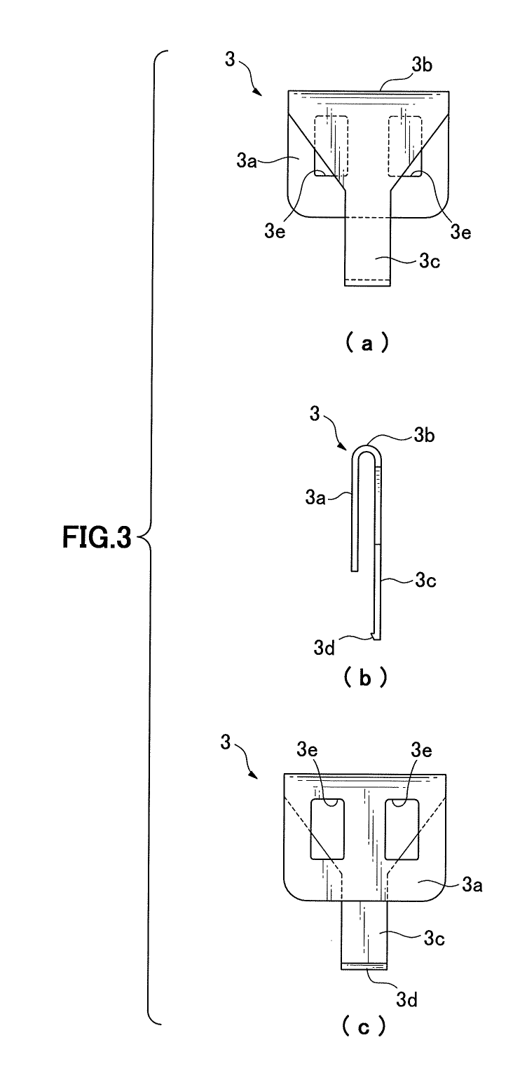

Firstly, referring to FIGS. 2(a) to 2(c) and FIGS. 3(a) to 3(c), a lumbar kyphosis-preventing lumbar brace according to a first embodiment of the present invention comprises an resilient resin plate 1 formed in a generally rectangular shape to serve as an resilient plate, and a belt engagement member 3 adapted to be attached to the resin plate 1. The resin plate is made of a resin material, such as a polypropylene resin, a high-pressure polyethylene resin or an EVA resin, and formed in a thin-pate shape which has a lateral width dimension W capable of covering a lateral region of a back surface of a lumbar portion of a wearer, and a vertical height dimension capable of covering between a hip region and a lower back region of the back surface of the lumbar portion of the wearer. The resin plate 1 is formed to have a thickness capable of being resilientally curved about at least a vertical axis thereof.

FIGS. 3(a) to 3(c) show a shape of the belt engagement member 3. The belt engagement member 3 has a rectangular-shaped belt engagement portion 3a, a bent portion 3b, and a leg portion 3c formed integrally with the belt engagement portion 3a through the bent portion 3b. The belt engagement member 3 may be made of the same resin material as that of the resin plate 1, or may be made of a resin material different from that of the resin plate 1. The leg portion 3c has a width less than that of the belt engagement portion 3a, and extends downwardly beyond a lower edge of the belt engagement portion 3a. The leg portion 3a has an engagement pawl 3d formed along an lower edge thereof to serve as a detect. The belt engagement portion 3a has two rectangular-shaped belt through-holes 3e formed therein with a distance therebetween in a widthwise direction thereof The belt engagement portion 3a may be formed in a hook-like structure hookable into the belt, to omit the belt through-holes 3e.

The resin plate 1 has three engagement-member mounting portions 1a formed in respective positions of a back surface thereof at certain intervals in the lateral direction to allow the belt engagement member 3 to be attached thereto. Each of the engagement-member mounting portions 1a is comprised of a low-height angular-C shaped protruding portion 1b formed integrally with the resin plate 1. In the embodiment illustrated in FIGS. 2(a) to 2(c), each of the engagement-member mounting portions 1a is comprised of a pair of the protruding portions 1b arranged with a distance therebetween in an upward-downward direction. As shown in FIG. 2(b), each of the protruding portions 1b defines a thin cross-sectionally rectangular-shaped engagement-member receiving hole 1c opened in the upward-downward direction. As shown in FIGS. 2(a) and 2(c), the leg portion of the belt engagement member 3 is inserted into the engagement-member receiving holes 1c from above an upper one of the protruding portions 1b. In the inserted state, the engagement pawl 3d formed along the lower edge of the leg portion 3c of the belt engagement member 3 is engaged with a lower edge of a lower one of the protruding portions 1b. This engagement makes it possible to prevent pull-out of the belt engagement member 3.

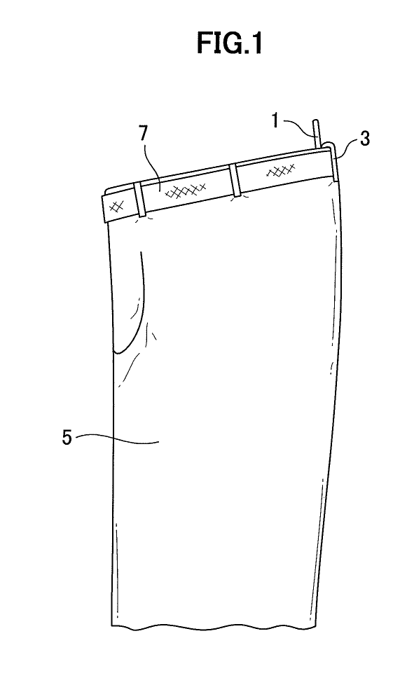

FIG. 1 shows a state after a lumbar kyphosis-preventing lumbar brace according to the present invention is attached to trousers. An ordinary type of trousers 5 has a belt 7. The lumbar kyphosis-preventing lumbar brace is placed in such a manner that the resin plate 1 is located inside the trousers 5, and the belt engagement member 3 is located outside the trousers 5. The belt 7 is inserted into the two rectangular-shaped belt through-holes 3e formed in the belt engagement portion 3a of the belt engagement member 3 to penetrate therethrough. In FIG. 2(a), the belt engagement member 3 is attached to only a central one of the engagement-member mounting portions 1a located in a widthwise central region of the resin plate 1. However, two additional belt engagement members 3 may be attached to respective ones of the engagement-member mounting portions 1a on both sides of the central engagement-member mounting portions 1a. In this case, the belt 7 is also inserted into the belt through-holes 3e of the additional belt engagement members 3 to penetrate therethrough.

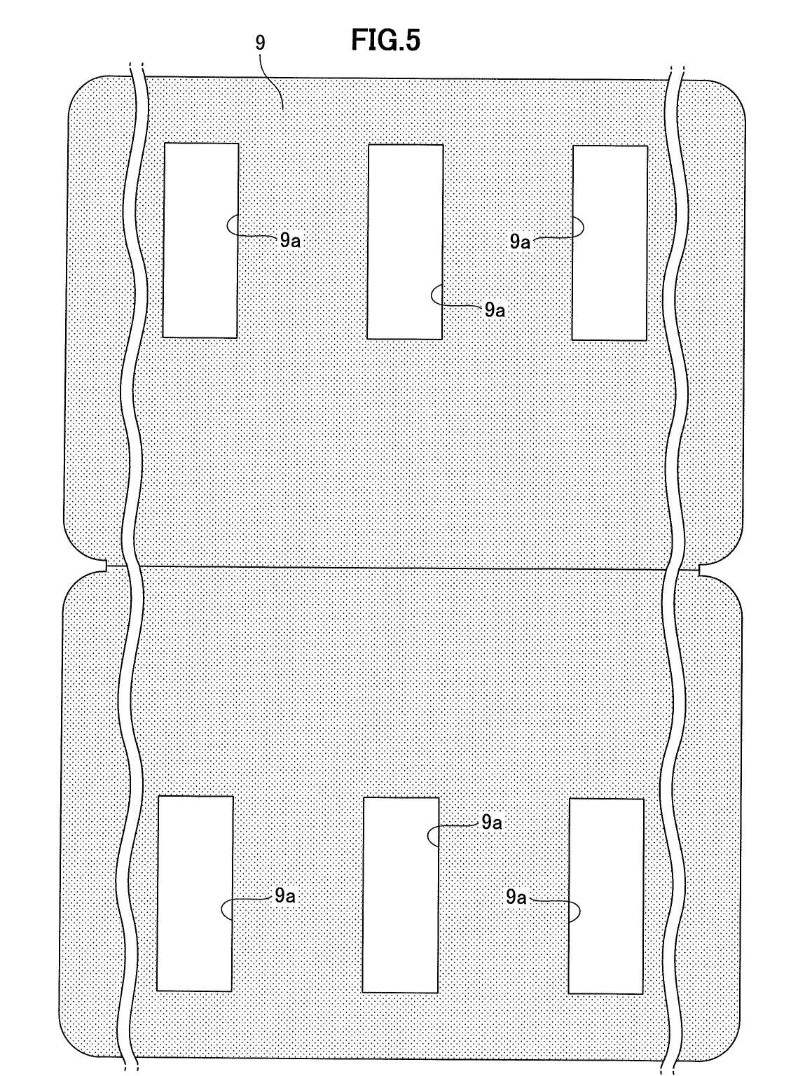

In the embodiment illustrated in FIGS. 2(a) to 2(c), a plurality of vent holes 1d are formed over the entire region of the resin plate 1 at even intervals. The vent holes 1d are effective in preventing wetting during use. In order to further improve wearability, a cloth 9, such as a nonwoven fabric cloth, may be attached onto at least a front surface of the resin plate 1 as one surface facing a wearer's body, preferably, onto both the front and back surfaces of the resin plate 1. FIG. 5 is a development view of the cloth 9 to be attached onto both the front and back surfaces of the resin plate 1, wherein six rectangular-shaped cutouts 9a are formed at respective positions corresponding to the engagement-member mounting portions 1a.

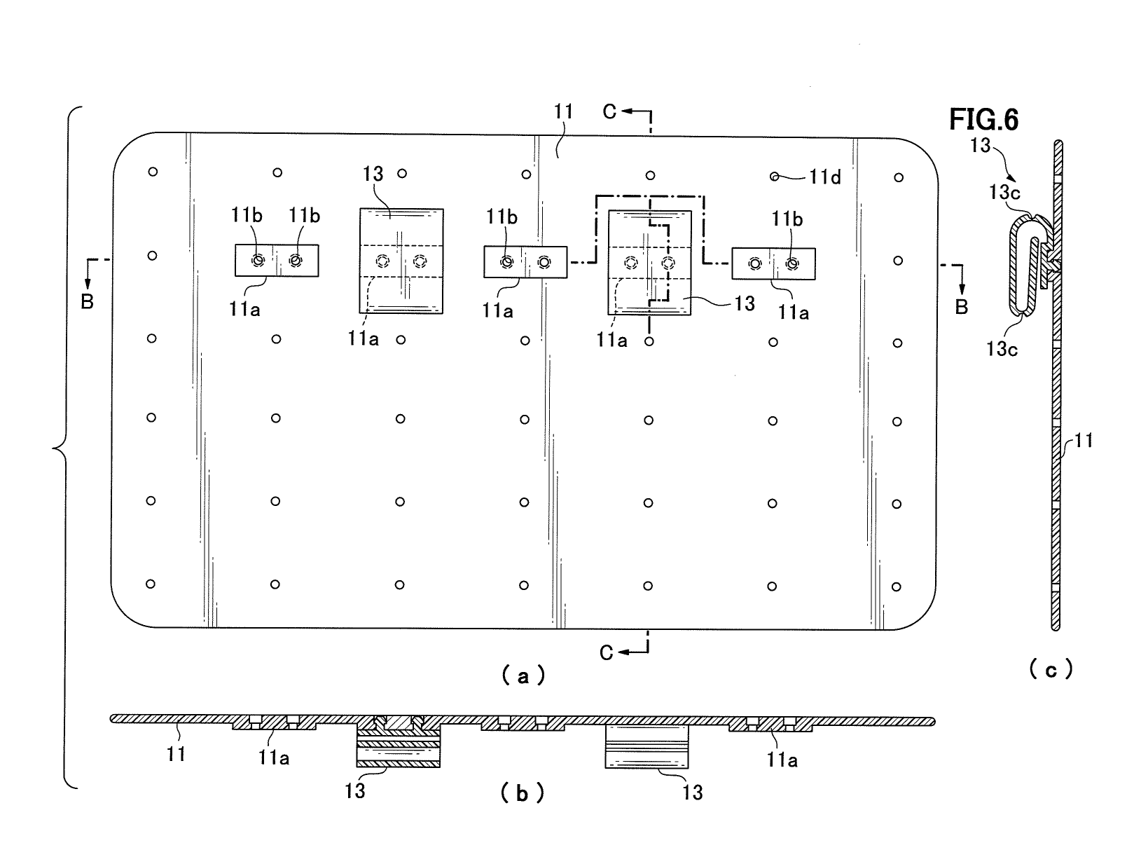

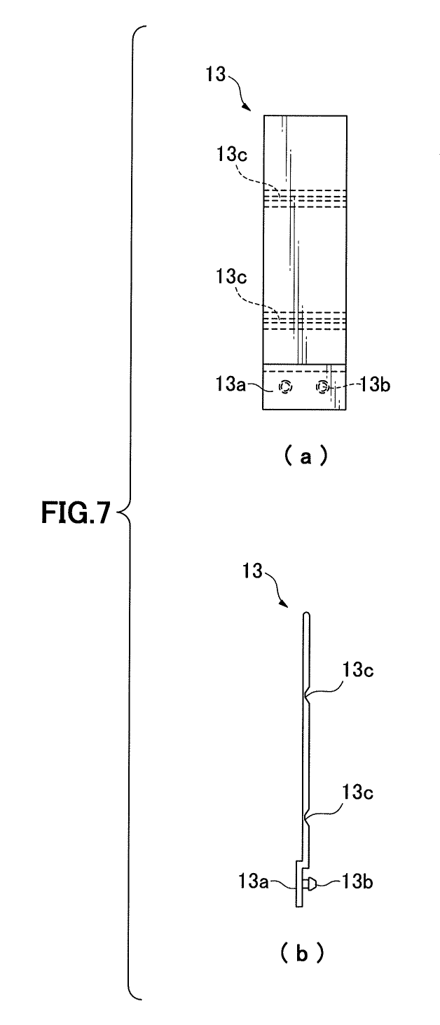

FIGS. 6(a) to 6(c) illustrate a lumbar kyphosis-preventing lumbar brace according to a third embodiment of the present invention. In FIGS. 6(a) to 6(c), a component or element corresponding to that in the embodiment illustrated in FIG. 2 is defined by a reference numeral determined by adding “10” to the corresponding reference numeral, and a duplicated description will be omitted. In the third embodiment, a belt engagement member 13 illustrated in FIGS. 7(a) and 7(b) is used. This belt engagement member 13 consists of an elongate strip-like member made of a resin material, and has a positioning leg portion 13a formed in one end thereof as a step-like depressed portion. This positioning leg portion 13a has a pair of pins 13b formed to protrude therefrom in side-by-side relation in a widthwise direction of the strip-like member. Further, the strip-like member as the belt engagement member 13 has a pair of hinge grooves 13c formed therein with a distance therebetween in a longitudinal direction thereof Each of the hinge grooves 13c is designed to allow the belt engagement member 13 to be bent along the hinge groove 13c while orienting the hinge groove 13c outwardly.

Referring to FIGS. 6(a) to 6(c), a resin plate 11 in the third embodiment has a plurality of mounting bases 11a formed to protrude toward a back surface thereof at certain intervals in a widthwise direction thereof. Each of the mounting bases 11a has a pair of engagement-member insertion holes 11b at respective positions corresponding to the pair of pins 13b formed on the belt engagement member 13. As shown in FIG. 6(c), the strip-like member is attached to the resin plate 11 in such a manner that it is bent into a ring shape along the hinge grooves 13c, and then the pins 13b are inserted into respective ones of the engagement-member insertion holes 11b. As seen in FIG. 6(c), the belt engagement member 13 defines a hole for allowing the belt 7 to penetrate therethrough.

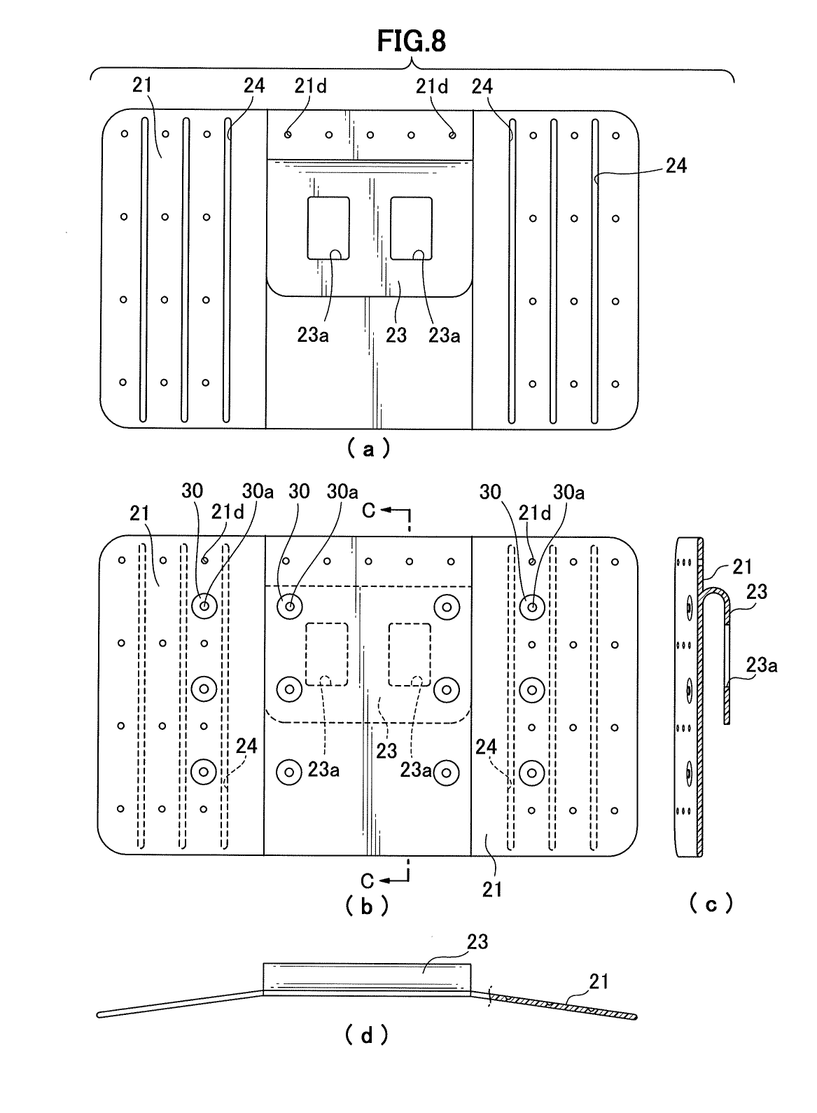

FIGS. 8(a) to 8(d) shows a lumbar kyphosis-preventing lumbar brace according to a fourth embodiment of the present invention. In FIGS. 8(a) to 8(d), a component or element corresponding to that in the embodiment illustrated in FIG. 2 is defined by a reference numeral determined by adding “20” to the corresponding reference numeral, and a duplicated description will be omitted. The lumbar brace according to the fourth embodiment has a structure in which a belt engagement member 23 is formed on a back surface of a resin plate 21 in integral relation with a resin plate 21. Further, a plurality of vertical grooves 24 are formed in the back surface of the resin plate 21. The vertical grooves 24 are intended to facilitate bending of the resin plate 21. The belt engagement member 23 has two belt through-holes 23a formed therein with a distance therebetween in a lateral direction thereof.

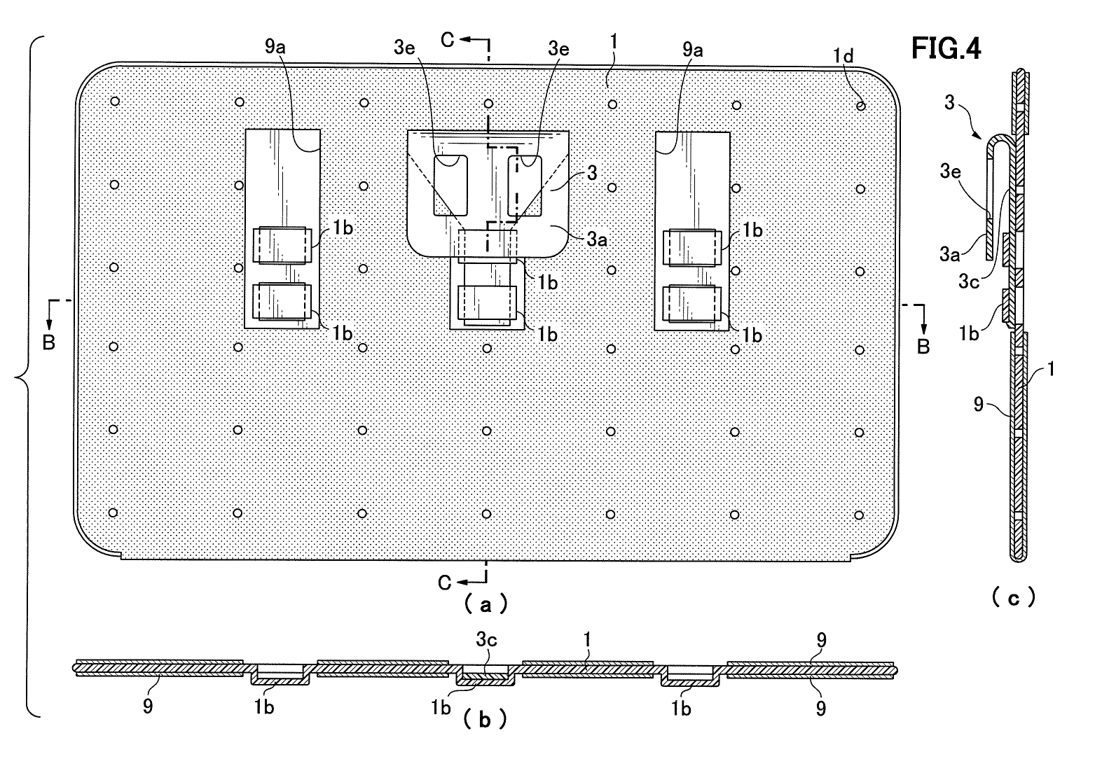

In the fourth embodiment, as shown in FIG. 8(b), a magnetic therapeutic device 30 having a permanent magnet piece 30a may be attached onto a surface of the resin plate 21 facing a wearer's body at an appropriate position. The magnetic therapeutic device 30 is designed to apply a magnetic field to a lumbar portion of the wearer to enhance a lower back pain-preventing effect. The magnetic therapeutic device 30 may also be provided on the resin plate in each of the first to fourth embodiments illustrated in FIGS. 2(a) to 7(b).

In the present invention, the resilient plate may be made of an animal hide such as a cattle hide, or a rubber material. In cases where the resilient plate is made of an animal hide, the material is preferably, but not limited to, a type having a tensile strength of 2.5 kg/mm² or more, and an elongation percentage of 16% or less under a load of 20 kg/mm² In the cases where the resilient plate is made of an animal hide or a rubber material, after material selection, dimensions such as thickness are determined to obtain resilientity enough to provide resistance to a forward bending (flexion) of a lumber portion of a wearer.

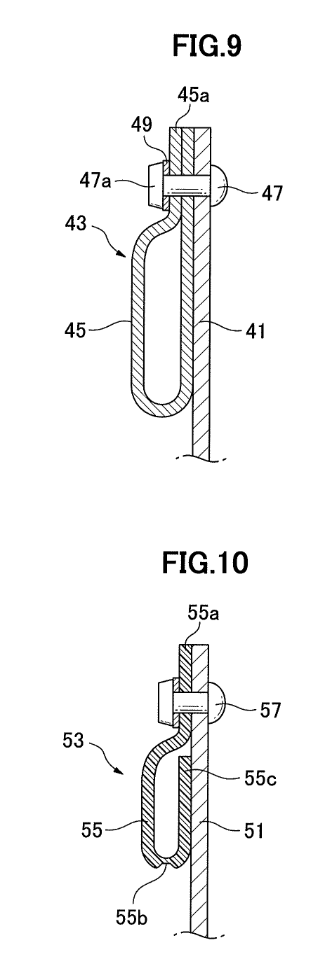

FIG. 9 is a sectional view showing another example of the belt engagement member. Although a belt engagement member in this example is suitable for use with the resilient plate made of an animal hide such as a cattle hide, or a rubber material, it may be used in combination with an resilient plate made of a different material such as a plastic material.

Referring to FIG. 9, the belt engagement member 43 is attached to a selected position of an resilient plate 41 made of a cattle hide. The belt engagement member 43 is formed by bending an elongate strip-like member 45 into a loop or ring shape, and joining opposite ends of the loop or ring-shaped strip-like member together by appropriate means such as sewing or bonding. The ring-shaped strip-like member 45 is attached to the resilient plate 41 by a rivet 47 through the joined ends 45a thereof. A washer 49 is provided between a crimped end 47a of the rivet 47 and the joined ends 45a of the strip-like member 45. In this attachment structure, the belt engagement member 43 is rotatable 360 degrees with respect to the resilient plate 41 to provide enhanced flexibility in use. In this example, the strip-like member 45 may be made of an animal hide, a rubber or a cloth. Preferably, the rivet 47 is an aluminum alloy rivet or a copper alloy rivet.

FIG. 10 shows yet another example of the belt engagement member. In this example, a belt engagement member 53 is made of a resin material such as a polypropylene resin, and attached to an resilient plate 51 made of an animal hide such as a cattle hide by a rivet 57. The belt engagement member 53 is comprised of an elongate strip-like resin plate 55, and the strip-like resin plate 55 is attached to the resilient plate 51 by the rivet 57 through one end 55a thereof. The strip-like resin plate 55 has a thin-walled portion 55b at an intermediate position thereof. The strip-like resin plate 55 is bent inwardly along the thin-walled portion 55b, and an end 55c of the bent portion is placed along a surface of the resilient plate 51.

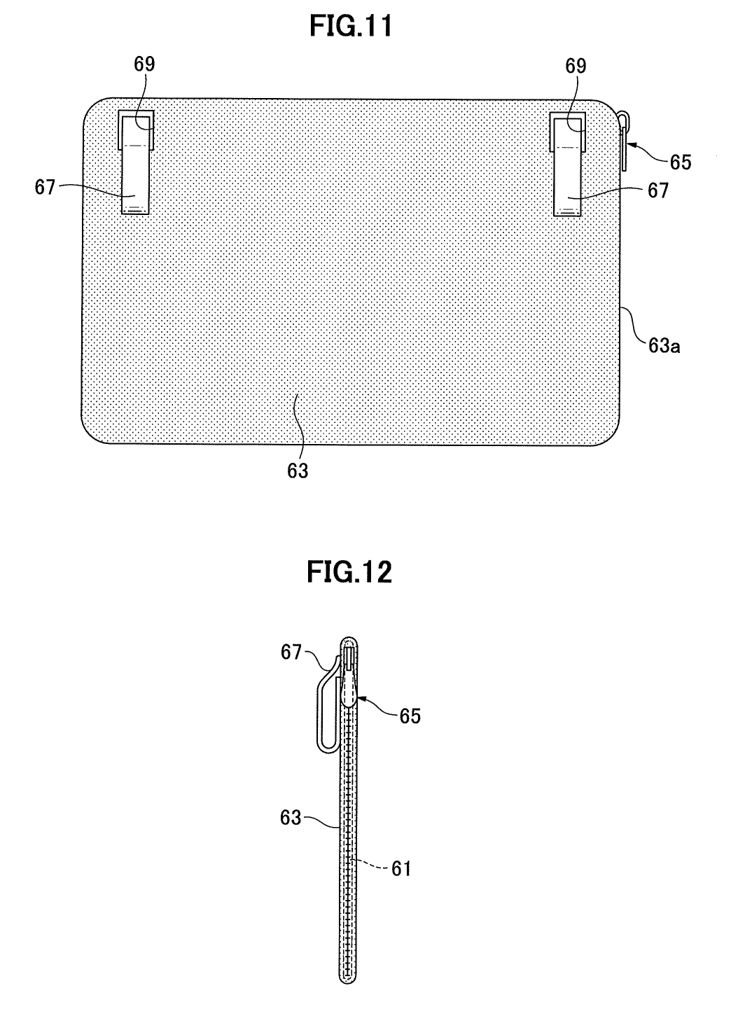

FIGS. 11 and 12 show a lumbar kyphosis-preventing lumbar brace according to a fifth embodiment of the present invention. If the resilient plate is made of an animal hide or a rubber material, an aesthetic appearance thereof is likely to become poor due to restrictions on tone. Thus, in the fifth embodiment, an resilient plate 61 is put in a bag-like breathable cloth cover 63. The cloth cover 63 has a structure in which at least one lateral edge 63a thereof is opened, and the open edge 63a is closed by a zipper fastener 65. The cloth cover 63 is formed with an opening 69 for allowing a belt engagement member 67 provided on the resilient plate 61 to penetrate therethrough.

1. A lumbar kyphosis-preventing lumbar brace comprising:
a generally rectangular-shaped resilient plate which has a width dimension capable of covering a lateral region of a back surface of a lumbar portion of a wearer, and a height dimension capable of covering between a hip region and a lower back region of the back surface of the lumbar portion; and
a belt engagement member provided on one side of the resilient plate in an engageable manner with a belt of a garment worn by the wearer,
wherein the lumbar brace is adapted to be worn in such a manner that the resilient plate is placed on the side of a wearer's body with respect to the garment, while allowing the belt engagement member to be located outside the garment and engaged with the belt of the garment.

2. The lumbar kyphosis-preventing lumbar brace as defined in claim 1, wherein the belt engagement member is formed integrally with the resilient plate.

3. The lumbar kyphosis-preventing lumbar brace as defined in claim 1, wherein the belt engagement member is formed separately from the resilient plate, and detachably attached to the resilient plate.

4. The lumbar kyphosis-preventing lumbar brace as defined in claim 3, wherein the resilient plate has an engagement member-receiving hole which is formed on the side of one surface thereof and opened in an upward-downward direction, and wherein the belt engagement member has a leg portion insertable into the engagement member-receiving hole.

5. The lumbar kyphosis-preventing lumbar brace as defined in claim 3, wherein the resilient plate has an engagement member-receiving hole formed to penetrate therethrough in a thicknesswise direction thereof, and wherein the belt engagement member has a leg portion formed with a pin insertable into the engagement member-receiving hole.

6. The lumbar kyphosis-preventing lumbar brace as defined claim 1, wherein the resilient plate has a plurality of vent holes formed to penetrate therethrough in a thicknesswise direction thereof.

7. The lumbar kyphosis-preventing lumbar brace as defined in claim 1, wherein the resilient plate has a cloth attached onto at least a surface thereof facing the wearer's body.

8. The lumbar kyphosis-preventing lumbar brace as defined in claim 1, wherein the resilient plate has one or more magnetic therapeutic devices each equipped with a permanent magnet piece and provided on a surface thereof facing the wearer's body.

9. The lumbar kyphosis-preventing lumbar brace as defined in claim 1, wherein the resilient plate is made of one or more selected from the group consisting of a resin material, a cattle hide and a rubber.

10. The lumbar kyphosis-preventing lumbar brace as defined in claim 1, wherein the belt engagement member comprises a strip-like member which is formed in a ring shape and attached to the resilient member by a rivet in a 360-degree rotation relation.

11. The lumbar kyphosis-preventing lumbar brace as defined in claim 1, wherein the resilient plate is put in a bag-like cloth cover having at least one lateral edge formed as a closable opening adapted to be closed by a zipper fastener.
